(19) United States
(12) Patent Application Publication
(10) Pub. No.: US 2022/0125454 A1
(43) Pub. Date: Apr. 28, 2022

(54) ACTUATED THROMBECTOMY DEVICE

(71) Applicant: Vicora, Inc., Buffalo, NY (US)

(72) Inventor: Richard Ducharme, Buffalo, NY (US)

(21) Appl. No.: 17/510,194

(22) Filed: Oct. 25, 2021

Related U.S. Application Data
(60) Provisional application No. 63/123,080, filed on Dec. 9, 2020.
(60) Provisional application No. 63/105,001, filed on Oct. 23, 2020.

(51) Int. Cl. A61B 17/22

(52) U.S. Cl. CPC A61B 17/22012; A61B 2017/00778

(57) ABSTRACT

A tip at the distal end of a catheter is designed to vibrates vigorously in order to break up a blood clot. Broken-up, the blood clot avoids “corking,” thus allowing it to be directly aspirated into the catheter. Unlike devices in minimally invasive surgery, where access to the organs to be removal are achieved through conveniently located small incisions, access to a location in the vascular space is achieved through a long flexible catheter, often 100 cm or more in length. An electroactive polymer (EAP) in at the tip of the distal end enables the vibration that breaks up the blood clot to be actuated from the proximal end of the catheter, without transferring mechanical action over substantially the entire length of the catheter.

### CROSS REFERENCE TO RELATED APPLICATIONS

The present application is related to and claims priority of (i) U.S. provisional application (“Provisional Application I”), Ser. No. 63/105,001, entitled “Actuated Thrombectomy Device,” filed on Oct. 23, 2020, and (ii) U.S. provisional application (“Provisional Application II”), Ser. No. 63/123,080, entitled “Actuated Thrombectomy Device,” filed on Dec. 9, 2020. The disclosures of Provisional Application I and Provisional Application II (collectively, “Provisional Applications”) are hereby incorporated by reference in their entireties.

### BACKGROUND OF THE INVENTION

### 1. Field of the Invention

The present invention relates to medical devices enabled by electroactive polymers (“EAP”; e.g., piezoelectric polymers). In particular, the present invention relates to a surgical instrument for thrombectomy based on an EAP.

### 2. Discussion of the Related Art

Heart attacks, pulmonary embolism, and stroke are among the examples of diseases caused by clots forming or migrating to a location within a blood vessel, and thereby clogging the blood vessel. These acute diseases are treated both pharmaceutically and by a mechanical procedure known as “mechanical thrombectomy.” For example, FIG. 1(a) shows a stentriever (e.g., the Solitaire X from Medtronic Neurovascular) being used to mechanically retrieve a blood clot. As another example, FIG. 1(b) shows removing a blood clot by direct aspiration through a catheter (e.g., the Penumbra system, available from Penumbra, Inc., Alameda, Calif.). Each of these techniques may be administered by itself or in combination with each other. Unfortunately, especially in strokes, the success rate in these procedures is poor, typically only 40-50% of the attempts result in the blood clots being removed. Thus, there is a significant need for improvements in both operational efficacy and favorable patient outcomes.

In a mechanical thrombectomy procedure, access to the blood clot is typically achieved using a catheter, which is typically about 100 cm long and which is threaded through a tortuous path through the vasculature. At the end of the procedure, the catheter is retracted along that same path in reverse. In aspiration, the blood clot frequently corks at the tip of the catheter, thus preventing it from being ingested into the catheter. Consequently, during retraction, the blood clot often breaks apart and may either (i) return to the original location, a condition known as “Embolism Distal Territory (EDT)”); or (ii) relocate to a new location, a condition known as “Embolism New Territory (ENT)”.

The current trend calls for the thrombectomy device to access even more distal locations within the vasculature. However, the typical mechanical thrombectomy device has become too bulky to track into blood vessels that are less than 2 mm in diameter. Likewise, aspiration catheters are also limited by its size. As the diameter of an aspiration catheter decreases to allow fitting into narrower blood vessels, at a constant aspiration pressure, the force the aspirator applies to the blood clot also drops quickly.

Thus, there is a long-felt need for a new mechanical thrombectomy device that overcomes the limitations of the aforementioned prior art devices.

### SUMMARY

According to one embodiment of the present invention, a catheter includes; (a) a proximal end configured for connection to a drive electronic circuit, so as to receive one or more electrical signals; (b) a distal end having a tip that an electroactive polymer actuator which is configured for vibrational motion in response to the electrical signals; and (c) a shaft coupled to the proximal end including wiring for carrying the electrical signals between the proximal end and the distal end. The electroactive polymer actuator may include a material including one or more of vinylidene fluoride (VDF), trifluoroethylene (TrFE), 1,1-chlorofluoroethylene (CFE), and chlorotrilfuoroethylene (CTFE). For example, the electroactive polymer actuator comprises a material including one or more of: P(VDF-TrFE-CTFE) and P(VDF-TrFE-CFE). The electroactive polymer actuator may exhibit an electrostrictive strain that is greater than 3% when the electrical signals provide an electric field of 20.0-200.0 volts per micron. The vibrational motion may have a frequency that is substantially tune to a resonant frequency of the tip.

In one embodiment, the shaft includes a non-conductive braid or coil in which the wiring is provided. The non-conductive braid or coil may be formed out of poly-tetrafluroethylene (PTFE) or poly-ether-ether ketone (PEEK). The distal end may further include an opening for ingesting by aspiration a blood clot broken up by the vibrational motion.

According to one embodiment of the present invention, the electroactive polymer actuator may include capacitors each including an electroactive polymer layer provided between a first electrode and a second electrode. The electroactive polymer layer may be between 2-20 um thick and formed by dip-coating in a solution of the electroactive polymer dissolved in a polar solvent (e.g., diethylformamide (DMF) or methyl ethyl ketone (MEK)). The electrodes may be formed by sputtering, dip-coating, pad printing or spray coating using a conductive electric ink.

According to another embodiment of the present invention, the first and second electrodes are braided to form space-apart coaxially placed coils. Each coil may be formed out of fine wire that has a 0.5-1.0 mils (i.e., thousandths of an inch) diameter. Alternatively, the first and second electrodes may be formed out of conductive wires in a Tri-Axe braid pattern.

According to yet another embodiment of the present invention, the electroactive polymer actuator may be one of numerous integrated actuators arranged in a three-dimensional array.

The present invention is better understood upon consideration of the detailed description below, in conjunction with the accompanying drawings.

### BRIEF DESCRIPTION OF THE DRAWINGS

FIG. 1(a) shows a stentriever being used to mechanically retrieve a blood clot.

FIG. 1(b) shows removing a blood clot by direct aspiration through a catheter.

FIG. 2(a) is a top view showing, at the distal end of catheter 100, vibratable tip 101 (“actuator”) and catheter shaft 104, according to one embodiment of the present invention.

FIG. 2(b) is a cross-section, transverse to the cross-section of FIG. 2(a), of actuator 101 at the distal end of catheter 100, showing electrode layers 108 and electroactive polymer layers 109.

FIG. 3 shows (conceptually) inner coil 201 at the distal end of catheter 100 connected to return electrode 106b in catheter shaft 104, according to one embodiment of the present invention.

FIG. 4(a) shows Tri-Axe wire 401 in Tri-Axe wire braid pattern 400.

FIG. 4(b) shows first and second sets of electrodes formed out of Tri-Axe wires in actuator 101 at the distal end of catheter 100, in accordance with one embodiment of the present invention.

FIGS. 5(a) and 5(b) represent cross-sectional and axial views of vibrational tip 101, respectively, in accordance with this embodiment of the present invention.

FIGS. 6(a) and 6(b) illustrate one method by which actuator 600 may be formed, according to one embodiment of the present invention.

### DETAILED DESCRIPTION OF THE PREFERRED EMBODIMENTS

The present invention provides an aspiration catheter that includes a tip at the distal end that vibrates vigorously to break up a blood clot. Broken-up, the blood clot avoids “corking,” thus allowing it to be directly aspirated into the catheter. Unlike devices in minimally invasive surgery, where access to the organs to be removal are achieved through conveniently located small incisions, access to a location in the vascular space is achieved through a long flexible catheter, often 100 cm or more in length. An electroactive polymer (EAP) in the tip at the distal end enables the vibration that breaks up the blood clot to be actuated from the proximal end of the catheter, without transferring mechanical action over substantially the entire length of the catheter. Suitable electroactive polymers include various combinations of vinylidene fluoride (VDF), trifluoroethylene (TrFE), 1,1-chlorofluoroethylene (CFE), and chlorotrifluoroethylene (CTFE). For example, the terpolymers P(VDF-TrFE-CTFE) and P(VDF-TrFE-CFE) are available commercially from Piezotech (a subsidiary of Arkema S.A., Paris, France). These terpolymers, which have different electroactive properties, exhibit large electrostrictive strain (>3%) under electric fields of 20-200 V/um (e.g., ˜50V/um).

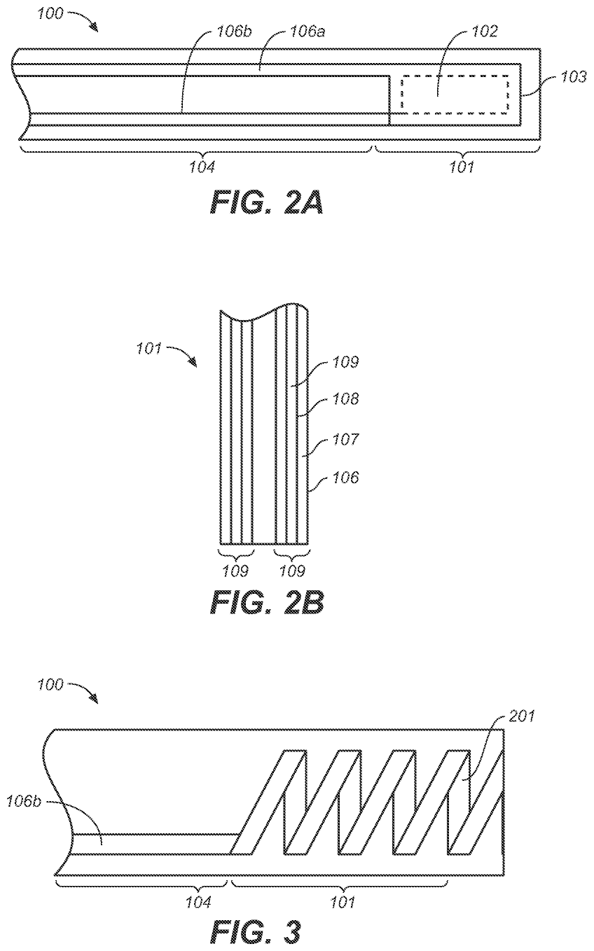

FIG. 2(a) is a top view showing, at the distal end of catheter 100, vibratable tip 101 and catheter shaft 104, according to one embodiment of the present invention. Vibratable tip 101 may be itself an actuator or includes one or more actuators that are each capable of electrically controlled motion. Catheter 100 includes a proximal end 105 (not shown) with a watertight connection to an electronic drive circuit to receive an electrical signal (e.g., 20-200 Hz), optimized to a resonant frequency of vibratable tip 101 at the distal end of catheter 100, so that it is suitable for both fracturing a blood clot and ingesting the debris of the blood clot by aspiration. For the intended operations, the electrical signal may have, for example, amplitudes 50.0-250.0 volts, with or without a DC offset.

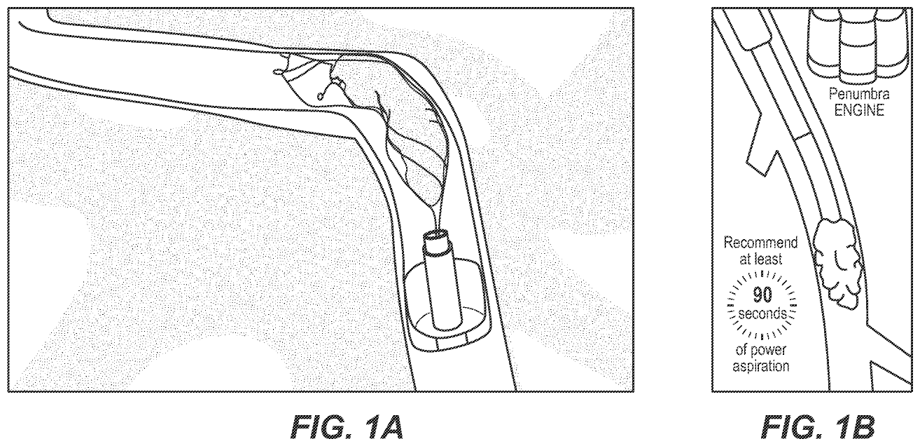

Catheter shaft 104 may be of conventional mechanical design, such as having an inner layer of poly-tetrafluroethylene (PTFE) in the form of a braid or coil, which provides catheter shaft 104 mechanical integrity and kink resistance. The PTFE inner layer may be surrounded by an outer layer of a reflowable material (e.g., Pebax with varying durometers across the length of catheter shaft 104). In addition, catheter shaft 104 accommodates both active electrode 106a and return electrode 106b, which are electrically insulated from each other, each extending along the entire length of catheter shaft 104. These electrodes may be formed out of any suitable electrically conductive wires. Such wires may be embedded in an electrically non-conductive braid or a coil (e.g., constructed from poly-ether-ether ketone (PEEK)) that extends along the entire length of catheter 100. These braids or coils are available in various patterns from, for example, Steeger USA, US Biodesign, Inc., and Admedes, Inc. Alternatively, an all-metallic braid with electrically insulated wires for active electrode 106a and return electrode 106b are also possible. However, embedding the electrodes in a non-conductive braid or coil is preferable to avoid shorting. Although, purely for illustrative purpose, only active electrode 106a and return electrode 106b are shown in FIG. 1, any suitable number of active electrodes and return electrodes may be used.

Vibratable tip 101 at the distal end of catheter 100 is configured for engaging a thrombus. Vibratable tip 101 has a flush or angled tip, so as to take maximal advantage of an opening through which the blood clot may be ingested. Layers of the EAP are embedded inside vibratable tip 101. Each EAP layer strains when an electric field is placed across it. (Note that, although a greater strain is achieved at a greater electric field, the strain-electric field relationship is generally non-linear.) As shown in FIG. 2(a), the EAP layers are each provided between thin and flexible layers of electrodes, e.g., between electrode 102 and electrode 103, which is underneath electrode 102. Electrodes 102 and 103 are each electrically connected to either active electrode 106a or return electrode 106b. In this manner, movement occurs only at vibratable tip 101 at the distal end of catheter 100 and no energy is lost in moving active electrode 106a and return electrode 106b catheter shaft. 104. In one embodiment, each EAP layer may be between 2-20 um thick.

According to one embodiment of the present invention, each EAP layer may be formed by dip-coating. For example, vibratable tip 101 at the distal end of catheter 100 may be dipped in a solution of the EAP in a polar solvent, such as diethylformamide (DMF) or methyl ethyl ketone (MEK). In this manner, coaxial 20-200 um thick EAP layers may be formed in vibratable tip 101 in successive dips. After forming each EAP layer, an electrode layer is formed over the exposed surface of the EAP layer by, for example, sputtering (e.g., gold or aluminum), clip-coating (e.g., silver-embedded urethane), pad printing or spray coating using a conductive electric ink or a particle-free metal-complex conductive ink (e.g. conductive inks available from Electroninks or LiquidX). The forming steps for the EAP layer-electrode layer combination may be repeated multiple times. The electrode layers thus formed may be connected to either active electrode 106a or return electrode 106b, such that electrodes of opposite polarities are formed on opposite sides of an EAP layer, creating effectively a capacitor. FIG. 2(b) is a cross-section, transverse to the cross-section of FIG. 2(a), of vibratable tip 101, showing electrode layers 108 and EAP layers 109. Depending on the mechanical properties desired, each EAP layer may have any one of various thicknesses. Additional non-EAP layers (not shown) may also be included.

According to another embodiment of the present invention, electrode layers in vibratable tip 101 at the distal end of catheter 100 may be braided to form two coaxially placed coils that are spaced apart to avoid electrical short. FIG. 3 shows (conceptually) inner coil 201 in vibratable tip 101 at the distal end of catheter 100 connected to return electrode 106b in catheter shaft 104, according to one embodiment of the present invention. Inner coil 201 is coaxially placed with and enclosed by outer coil 202 connected to active electrode 106a. FIG. 3 is a conceptual depiction, showing only for purely illustrative purpose six turns of a single wire in inner coil 201. In a realistic implementation, a braided coil of many more turns and many more wires are expected. For example, a braided coil of up to 288 wires in sizes down to (0.0005″×0.002″) for flat wire and 0.0005″ for round wire. (See, e.g., https://steegerusa.com/product/medical-braiders, available from Steeger USA.) An EAP can be coated over and fills the space between inner coil 201 and outer coil 202, such that an electric field is created in that space when a voltage difference is established between the coils. Each coil may be formed out of fine wire that has a 0.5-1.0 mils (i.e., thousandths of an inch) diameter. This embodiment has the advantage of a reduced manufacturing time, requiring only a single application or dip of the EAP and simplifies the electrode connections, using wiring that is already provided through the entirety of catheter 100.

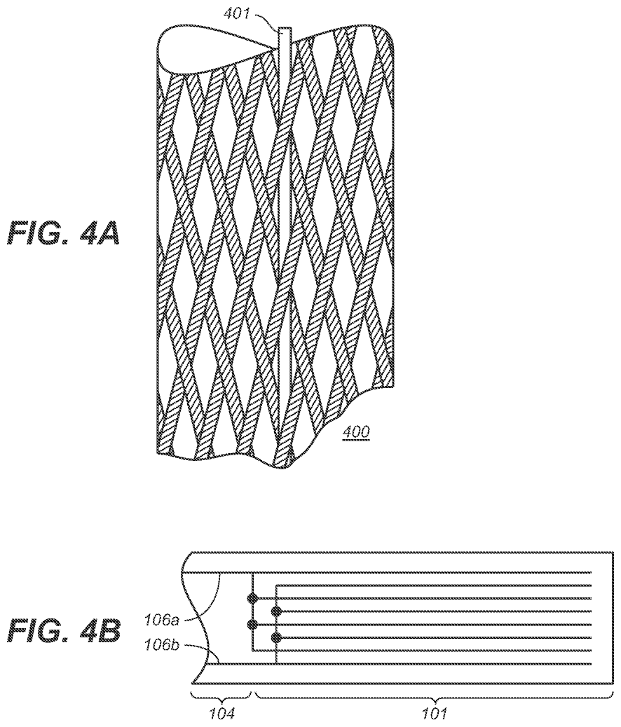

According to a third embodiment of the present invention, the electrodes in vibratable tip 101 may be provided in vibratable tip 101 at the distal end of catheter 100 by “Tri-Axe” wires in a Tri-Axe braid pattern. FIG. 4(a) shows Tri-Axe wire 401 in Tri-Axe wire braid pattern 400. A Tri-Axe wire braid pattern consists of single wires (e.g., wire 401 being one) routed straight enclosed within the Tri-Axe braid pattern (e.g., Tri-axe braid pattern 400). As shown in FIG. 4(a), in Tri-Axe wire brain pattern 400, the Tri-Axe wires themselves (e.g., tri-axe wire 401) are not braided. Such Tri-Ax wires can be used up to half the capacity of a full load, thus providing many “Tri-axe” wires that can be integrated and used as electrodes. Generally, the greater the number of wires and the smaller the size, the better the electromechanical response. FIG. 4(b) shows first and second sets of electrodes formed out of Tri-Axe wires in vibratable tip 101 at the distal end of catheter 100, in accordance with one embodiment of the present invention. The remainder of the Tri-Axe braid pattern is omitted from FIG. 4(b). The first and second sets of electrodes may be provided by round or flat wires each as thin as 0.5 mils, providing up to 288 electrodes.

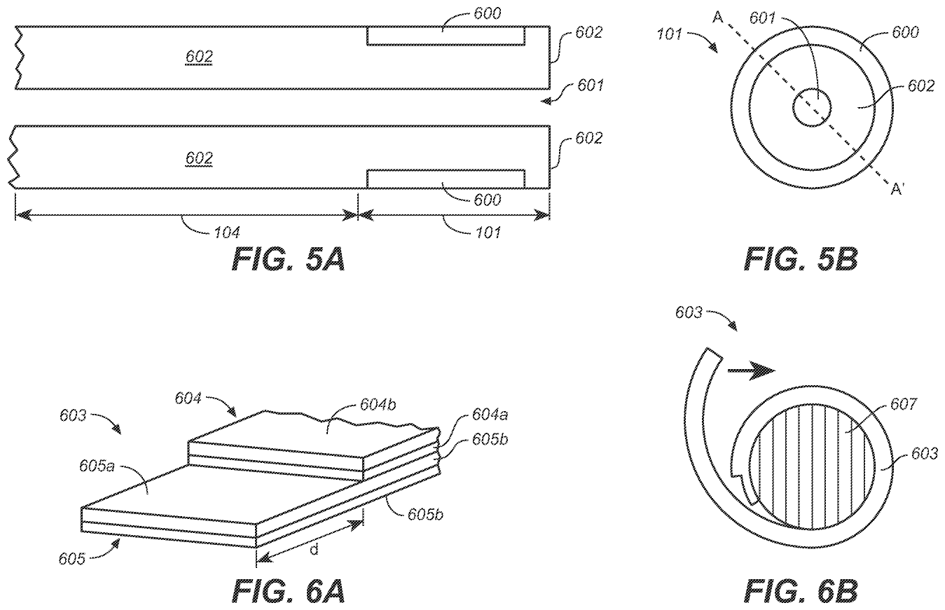

In the embodiments described above, the electrodes and the EAP layer or layers are individually provided. According to one embodiment of the present invention, however, there are EMP actuators (“integrated actuators”) that are commercially available. These integrated actuators have characterized electromechanical properties and may be rolled into any desired geometry for deployment in vibratable tip 101 at the distal end of catheter 100. Thus, one or more integrated actuators may be incorporated into vibratable tip 100 (e.g., as a three-dimensional array of integrated actuators) at the distal end of catheter 100. FIG. 5 shows a commercially available EAP actuator. Each such actuator may function at the same, or different frequencies or patterns.

Each of the embodiments described above may be driven by a drive electronic circuit. if vibratable tip 101 is designed to have multiple independently controlled actuators, more than one waveform may be provided to each of the active electrodes. In most embodiments described above, the drive circuit may provide driving waveforms, for example, between 50.0-250.0 volts (peak-to-peak). The driving waveform may be sinusoidal, triangular, square or any desired wave shape (preferably, a square wave) to provide the greatest acceleration or vibration. A suitable driving circuit may be provided, for example, using Microchip HV56020 or Microchip HV 56022.

According to another embodiment of the present invention, vibrational tip 101 of catheter 100 may include an actuator formed out of two or more layers of EAP films wrapped around a recess in a cylindrical shaft. FIGS. 5(a) and 5(b) represent cross-sectional and axial views of vibrational tip 101, respectively, in accordance with this embodiment of the present invention. As shown in FIG. 5(a), catheter 100 includes lumen 601, which extends along catheter 100's axis substantially its entire length. FIG. 5(a) is a cross section through the longitudinal axis of catheter 100, showing a portion of catheter shaft 104 and vibrational tip 101. Catheter material 602 in catheter shaft 104 extends into vibrational tip 101. Over a section that is 2.0-5.0 mm long along the longitudinal axis in vibrational tip 101, catheter material 602 is shrunk to a lesser diameter to provide a recess in vibrational tip 101. Actuator 600, which is tubular (i.e., cylindrical with a hollow core) with an annular cross section, is attached, wrapped around or otherwise integrated in the recess. FIG. 5(b) is an axial view at an orthogonal planar cross section through vibrational tip 101, showing actuator enclosing catheter material 602, which in turn encloses lumen 601.

FIGS. 6(a) and 6(b) illustrate one method by which actuator 600 may be formed, according to one embodiment of the present invention. As shown in FIG. 6(a), EAP film 604 is overlaid on top of EAP film 605, offset by a short distance (d), to form composite sheet 603. On one side of each film is coated conductive material (e.g., a metallic coating, such as a copper film). In composite sheet 603, EAP film 604 includes EAP material 604a and conductive coating 604b. Likewise, EAP film 605 includes EAP material 605a and conductive coating 605b. EAP materials 604a and 605b may each be, for example, a terpolymer. As shown in FIG. 6(a), conductive coatings 604b and 605b are provided on mutually obverse sides of composite sheet 603, thereby providing an EAP layer—consisting of EAP materials 604a and 604b—between conductive coatings 604b and 605b, in a parallel-plate capacitor configuration. In this arrangement, conductive coatings 604b and 605b are positioned on the outside of composite sheet 603 to allow them to serve as electrodes for composite sheet 603, allowing composite sheet 603 to receive signals over electrical leads that may be provided in lumen 601. These electrical leads electrically connect composite sheet 603 to an electronic or electrical circuit provided at distal end 105 of catheter 100.

To form actuator 600, composite sheet 603 may be wrapped around cylindrical mandril 607 multiple times, as illustrated in FIG. 6(b). (In these FIGS. 6(a) and 6(b), the thickness of composite sheet 603 is exaggerated to distinctly show EAP materials 604a and 605a and conductive coatings 604b and 605b; in a practical implementation, composite sheet 603 can be made very thin (e.g., a few tenths of microns or a few millimeters), so that composite sheet 603 may be wrapped around mandril 607 many times, thus providing a large surface area (i.e., as compact form) for greater control of composite sheet 603's electromechanical response.) Mandril 607 can then be withdrawn, thus leaving actuator 600 in a cylindrical form with a hollow core. Actuator 600 can then be mounted onto the recess in vibrational tip 101 of catheter 100. Electrical leads can then be attached to exposed electrical coatings 604a and 604b to electrically connect composite film 603 to a control circuit that may be provided at the distal end of catheter 100. Offset d in composite sheet 603 facilitates the attachment.

When a voltage is applied across conductive layers 604b and 605b of composite sheet 603 in actuator 600, the EAP material in EAP materials 604a and 605a expands or contracts volumetrically (i.e., a strain response), which provides actuator 600's circumferential strain response. Consequently, a sequence of electrical pulses (e.g., a square wave) at an appropriate frequency (e.g., 20.0-500.0 Hz) may generate a desirable circumferential vibration in vibratable tip 101. Note that, the direction of the polarization makes little or no difference in device performance, as a waveform alternating between −50.0 volts to 50.0 volts provide substantially the same electromechanical response in actuator 600 as a waveform alternating between 0.0 volts and 50.0 volts, for any given frequency. Any high slew-rate waveforms that provide a rapidly changing electric field across conductive coatings 504b and 504d can also be used.

The above detailed description is provided to illustrate specific embodiments of the present invention and is not intended to be limiting. Numerous variations and modifications within the scope of the present invention are possible. The present invention is set forth in the accompanying claims.

I claim:

1. A catheter, comprising;
a proximal end configured for connection to a drive electronic circuit, so as to receive one or more electrical signals;
a distal end having a tip that comprises an electroactive polymer actuator which is configured for vibrational motion in response to the electrical signals; and
a shaft coupled to the proximal end including wiring for carrying the electrical signals between the proximal end and the distal end.

2. The catheter of claim 1, wherein the electroactive polymer actuator comprises a material including one or more of vinylidene fluoride (VDF), trifluoroethylene (TrFE), 1,1-chlorofluoroethylene (CFE), and chlorotrifluoroethylene (CTFE).

3. The catheter of claim 1, wherein the electroactive polymer actuator comprises a material which includes one or more of: P(VDF-TrFE-CTFE) and P(VDF-TrFE-CFE).

4. The catheter of claim 1, wherein the electroactive polymer actuator exhibits an electrostrictive strain that is greater than 3% when the electrical signals provide an electric field of 20-200 volts per micron.

5. The catheter of claim 1, wherein the vibrational motion has a frequency that is substantially tune to a resonant frequency of the tip.

6. The catheter of claim 1, wherein one of the electrical signals has an amplitude between 50 volts and 250 volts,

7. The catheter of claim 6, wherein one of the electrical signals has a DC offset.

8. The catheter of claim 1, wherein the shaft includes a non-conductive braid or coil in which the wiring is provided.

9. The catheter of claim 8, wherein the non-conductive braid or coil is formed out of poly-tetrafluroethylene (PTFE) or poly-ether-ether ketone (PEEK)

10. The catheter of claim 1, wherein the tip in the distal end further comprises an opening for ingesting a blood clot broken up by the vibrational motion.

11. The catheter of claim 10, configured to be connected to an aspirator to provide a pressure for ingesting the blood clot.

12. The catheter of claim 1, wherein the electroactive polymer actuator comprises a plurality of capacitors each including an electroactive polymer layer provided between a first electrode and a second electrode.

13. The catheter of claim 12, wherein the electroactive polymer layer is between 2.0-20.0 um thick.

14. The catheter of claim 12, wherein the electroactive polymer layer is formed by dip-coating in a solution of the electroactive polymer dissolved in a polar solvent.

15. The catheter of claim 14, wherein the polar solvent comprises one or more of diethylformamide (DMF) and methyl ethyl ketone (MEK).

16. The catheter of claim 12, wherein each of the first and second electrodes comprises a material formed by sputtering, dip-coating, pad printing or spray coating using a conductive electric ink.

17. The catheter of claim 12, wherein the first and second electrodes are braided to form space-apart coaxially placed coils.

18. The catheter of claim 17, wherein each coil is formed out of fine wire that has a 0.5-1.0 mils (i.e., thousandths of an inch) diameter.

19. The catheter of claim 12, wherein each of the first and second electrodes comprise conductive wires in a Tri-Axe braid pattern.

20. The catheter of claim 1, wherein the electroactive polymer actuator is one of a plurality of integrated actuators arranged in a three-dimensional array.

21. The catheter of claim 1, wherein one of the electrical signals is sinusoidal.

22. The catheter of claim 1, wherein one of the electrical signals has a square waveform.

23. The catheter of claim 22, wherein the electroactive polymer actuator comprises two or more layers of EAP material rolled into a compact form.

24. The catheter of claim 23, wherein the electroactive polymer actuator is formed as a cylindrical structure with a hollow core.

25. The catheter of claim 23, wherein each layer of EAP material is coated on one side by a conductive material.

26. The catheter of claim 25, wherein the conductive material comprises a metal.

27. The catheter of claim 25, wherein the electroactive polymer actuator is provided a parallel-plate capacitor configuration, with electrodes being provided by the conductive coatings.

28. The catheter of claim 27, wherein the electroactive polymer actuator provides a vibrational response when actuated by an electrical signal of a frequency between 20.0-500.0 Hz.

29. The catheter of claim 27, wherein the electrical signal comprises a high slew rate waveform.

30. The catheter of claim 27, wherein the waveform has a peak-to-peak amplitude between 50.0-250.0 volts.
